(19) United States
(12) Patent Application Publication
(10) Pub. No.: US 2018/0103941 A1
(43) Pub. Date: Apr. 19, 2018

(54) SURGICAL RETRACTOR

(71) Applicant: Agha Khan, Lutherville, MD (US)

(72) Inventor: Agha Khan, Lutherville, MD (US)

(21) Appl. No.: 15/292,346

(22) Filed: Oct. 13, 2016

(51) Int. Cl. A61B 17/02

(52) U.S. Cl. CPC A61B 17/025; A61B 2017/00407; A61B 2017/0256; A61B 17/0218

(57) ABSTRACT

A surgical retractor includes a pair of handles. Each handle includes a handle shaft that extends along a shaft axis lying in a first plane. A retractor arm extends from each handle shaft. Each retractor arm includes a first portion that extends along the first plane along a first axis tilted at a first angle with respect to the respective handle shaft, a second portion that extends along a second axis tilted at a second angle with respect to the first portion, and a third portion that extends along a third axis lying in a second plane. A retractor blade extends from each of the retractor arms. Each retractor blade includes a spine that extends along a fourth axis lying in a third plane. An end of the spine extends from the third portion. Fingers extend away from the spine.

### FIELD OF THE INVENTION

The present invention relates generally to surgical retractors, and particularly to surgical retractors that provide maximum exposure with minimal incision, such as for lumbar spinal surgery.

### BACKGROUND OF THE INVENTION

To obtain a good exposure in any surgery while keeping the incision size minimal is always challenging. This is perhaps more challenging in lumbar spinal surgery. An ideal retractor in lumbar spinal surgery, especially open surgery, would need to be able to:

1) Provide maximum exposure with minimal incision.

2) Be out of the way of the surgeon.

3) Be atraumatic to the tissue.

4) Allow the surgeon to utilize the maximum length of the incision.

Lots of retractors of various types have been designed but they fail to meet the above criteria for various reasons, such as bulkiness, need for long incision and other factors.

### SUMMARY OF THE INVENTION

The present invention seeks to provide novel surgical retractors, as is described more in detail herein below.

In one embodiment, the retractor arms have multiple angulation, which makes it possible to reach corners of longitudinal incisions and thus increases the exposure and illumination of the surgical incision. The handles of the retractor are ratcheted and the handles lie flat on the outer surface of the patient such that the handles do not get in the way of the surgeon. In another embodiment, the retractors have an offset blade that is helpful in retracting tissue in a paramedian approach.

There is thus provided in accordance with an embodiment of the present invention a surgical retractor including a pair of handles, each of the handles including a handle shaft that extends along a shaft axis lying in a first plane, a retractor arm extending from each of the handle shafts, each retractor arm including a first portion that extends along the first plane along a first axis tilted at a first angle with respect to the respective handle shaft, a second portion that extends along the first plane along a second axis tilted at a second angle with respect to the first portion, a third portion that extends along a third axis lying in a second plane, the second plane being tilted at a third angle with respect to the first plane, and a retractor blade extending from each of the retractor arms, each of the retractor blades including a spine that extends along a fourth axis lying in a third plane, the third plane being tilted at a fourth angle with respect to the second plane, an end of the spine extending from the third portion, and the retractor blade further including fingers that extend away from the spine.

In accordance with an embodiment of the present invention the fingers extend perpendicular to the spine along the third plane.

In accordance with an embodiment of the present invention each of the fingers includes a curved distal tip.

In accordance with an embodiment of the present invention the handles are ratcheted with respect to a pivot point by means of a ratchet mechanism.

The end of the spine that extends from the third portion may or may not be adjacent one of the fingers. In the event it is not adjacent one of the fingers, this means the spine has a portion free of fingers and the fingers are positioned more distally to provide deeper or wider retraction capability.

### BRIEF DESCRIPTION OF THE DRAWINGS

The present invention will be understood and appreciated more fully from the following drawings:

FIGS. 1A-1C are simplified perspective, top-view and side view illustrations, respectively, of a surgical retractor, constructed and operative in accordance with a non-limiting embodiment of the present invention, with the retractor in an open position;

FIGS. 2A-2C are simplified perspective, top-view and side view illustrations, respectively, of the surgical retractor in a closed position; and

FIG. 3 is a simplified pictorial illustration of a surgical retractor, constructed and operative in accordance with another non-limiting embodiment of the present invention.

### DETAILED DESCRIPTION OF EMBODIMENTS

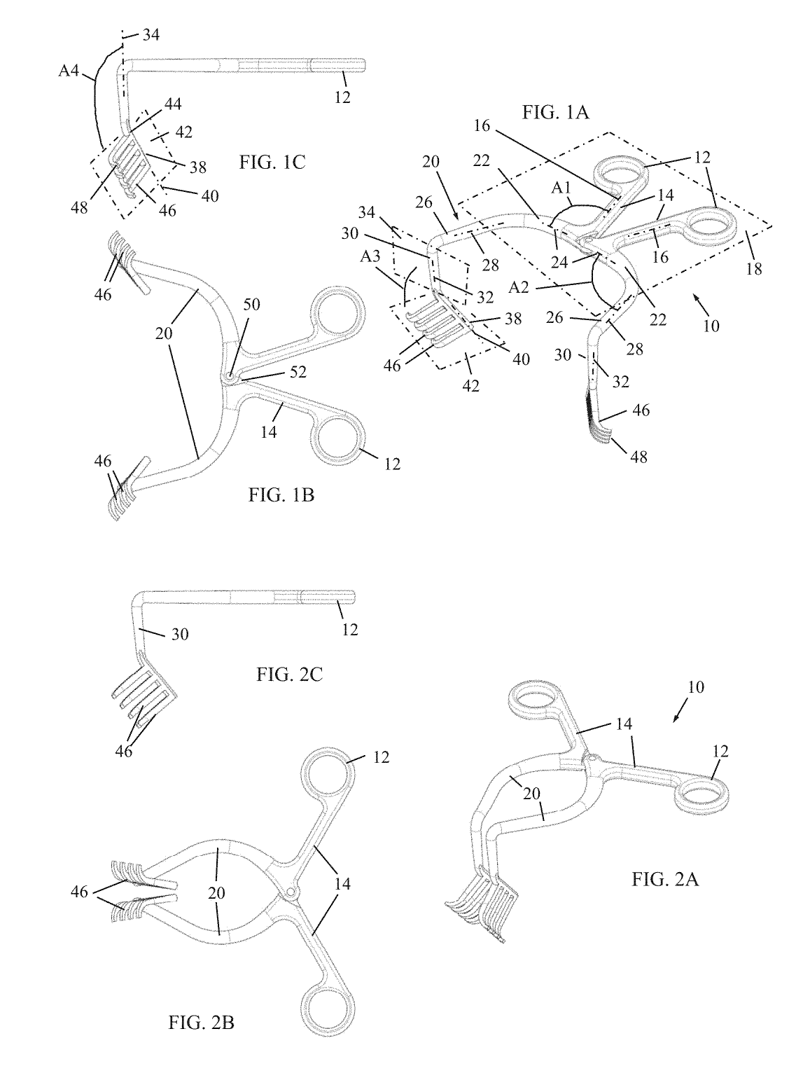

Reference is now made to FIGS. 1A-1C, which illustrate a surgical retractor 10, constructed and operative in accordance with a non-limiting embodiment of the present invention.

Surgical retractor 10 includes a pair of handles 12. Each handle 12 includes a handle shaft 14 that extends along a shaft axis 16 lying in a first plane 18.

A retractor arm 20 extends from each handle shaft 14. Each retractor arm includes a first portion 22 that extends along the first plane 18 along a first axis 24 tilted at a first angle A1 with respect to the respective handle shaft 14. In one non-limiting embodiment, first angle A1 is between 0-90°. A second portion 26 of retractor arm 20 extends along the first plane 18 along a second axis 28 tilted at a second angle A2 with respect to the first portion 22. In one non-limiting embodiment, second angle A2 is between 90-180°. A third portion 30 of retractor arm 20 extends along a third axis 32 lying in a second plane 34. Second plane 34 is tilted at a third angle A3 with respect to first plane 18. In one non-limiting embodiment, third angle A3 is between 0-180°.

A retractor blade 36 extends from each retractor arm 20. Each retractor blade 36 includes a spine 38 that extends along a fourth axis 40 lying in a third plane 42. The third plane 42 is tilted at a fourth angle A4 with respect to the second plane 34. In one non-limiting embodiment, fourth angle A4 is between 0-180°. An end 44 of spine 38 extends from the third portion 30 of retractor arm 20.

The retractor blade 36 further includes fingers 46 that extend away from spine 38. The fingers 46 may extend perpendicular to spine 38 along the third plane 42. In accordance with an embodiment of the present invention each finger 46 includes a curved distal tip 48.

In accordance with an embodiment of the present invention the handles 12 are ratcheted with respect to a pivot point 50 by means of a ratchet mechanism 52 (e.g., a pawl and teeth mechanism).

FIGS. 2A-2C illustrate the surgical retractor 10 in a closed position. It is noted that the multiple angulation of the retractor arms 20 of surgical retractor 10 enable the surgeon to reach corners of longitudinal incisions and thus increases the exposure and illumination of the surgical incision. The surgical retractor 10 can lie flat on first plane 18, which is the resting surface of the retractor on the outside surface of the patient. Handles 12 do not get in the way of the surgeon, and the fingers can reach deep down for good retraction and gripping.

In the embodiment of FIGS. 1A-1C, the end 44 of the spine 38 that extends from the third portion 30 may be adjacent one of the fingers 46.

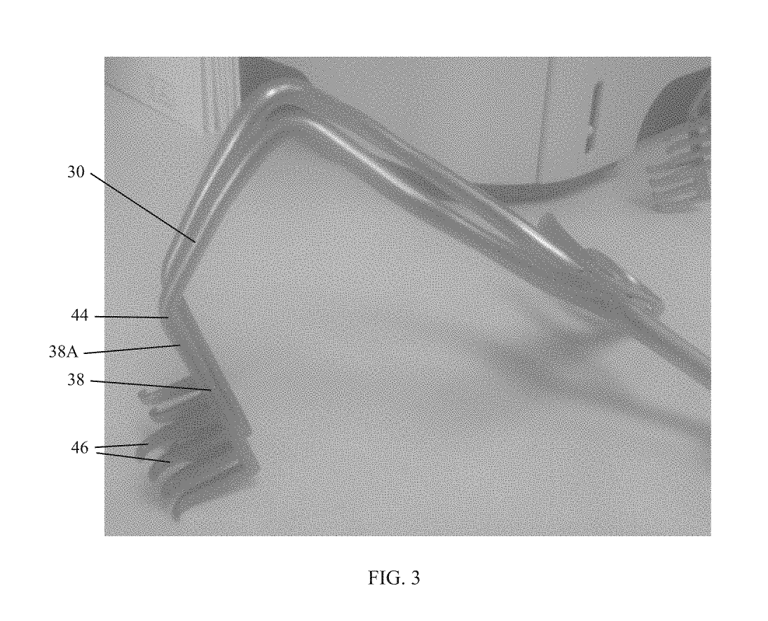

Reference is now made to FIG. 3. In this embodiment, the end 44 of the spine 38 that extends from the third portion 30 is not adjacent one of the fingers 46. In other words, the spine 38 has a portion 38A free of fingers and the fingers 46 are positioned more distally to provide deeper or wider retraction capability.

What is claimed is:

1. A surgical retractor comprising:
a pair of handles, each of said handles comprising handle shaft that extends along a shaft axis lying in a first plane;
a retractor arm extending from each of said handle shafts, each retractor arm comprising a first portion that extends along said first plane along a first axis tilted at a first angle with respect to the respective handle shaft, a second portion that extends along said first plane along a second axis tilted at a second angle with respect to said first portion, a third portion that extends along a third axis lying in a second plane, the second plane being tilted at a third angle with respect to said first plane; and
a retractor blade extending from each of said retractor arms, each of said retractor blades comprising a spine that extends along a fourth axis lying in a third plane, the third plane being tilted at a fourth angle with respect to said second plane, an end of said spine extending from said third portion, and said retractor blade further comprising fingers that extend away from said spine.

2. The surgical retractor according to claim 1, wherein said fingers extend perpendicular to said spine along said third plane.

3. The surgical retractor according to claim 1, wherein each of said fingers comprises a curved distal tip.

4. The surgical retractor according to claim 1, wherein said handles are ratcheted with respect to a pivot point by means of a ratchet mechanism.

5. The surgical retractor according to claim 1, wherein the end of said spine that extends from said third portion is adjacent one of said fingers.

6. The surgical retractor according to claim 1, wherein the end of said spine that extends from said third portion is not adjacent said fingers.
